(19) United States
(12) Patent Application Publication
(10) Pub. No.: US 2025/0387039 A1
(43) Pub. Date: Dec. 25, 2025

(54) METHOD FOR ANALYSING THE SWEAT PRODUCED BY THE SKIN OF A USER AND ANALYSIS DEVICE FOR IMPLEMENTING SUCH A METHOD

(71) Applicant: BE-LAB, WAMBRECHIES (FR)

(72) Inventor: Gérard-Marie PAPIEROK, HYERES (FR)

(21) Appl. No.: 18/879,537

(22) PCT Filed: Jun. 29, 2023
(86) PCT No.: PCT/IB2023/056755

§ 371 Date: Dec. 27, 2024

(30) Foreign Application Priority Data
Jun. 30, 2022 (FR) FR2206659

(51) Int. Cl. A61B 5/053; A61B 5/00

(52) U.S. Cl. CPC A61B 5/053; A61B 5/4266; A61B 5/681; A61B 5/6831

(57) ABSTRACT

Disclosed is a method for analyzing sweat produced by the skin of a user including a first step of collecting sweat released by the skin by a collector of a housing to form a continuous flow of sweat of a second face of the housing, which is provided with a microfluidic channel. The method includes a third step of measuring a first conductance of the continuous flow of sweat by a first pair of electrodes housed inside the microfluidic channel and a fourth step of measuring a second conductance of the continuous flow of sweat by a second pair of electrodes located at an electrode distance from the first pair of electrodes. The method includes a fifth step of determining a period of time taken for the second conductance to become equal to the first conductance and a sixth step of calculating a flow rate of the sweat.

The field of the present invention is that of the methods for analyzing a sweat emitted by a skin of a user. The object thereof is such a method for analyzing a sweat emitted by a skin of a user as well as an analysis device for implementing such a method.

The document WO 2018/017619 describes a device for analyzing a sweat of a user. The device is configured to measure conductivity of the sweat, a cutaneous conductance of the skin and a volumetric perspiration rate. For this purpose, the device is intended to be placed on a skin of the user and the device comprises a sensor of conductivity of the sweat, a sensor of cutaneous conductance and a sensor of volumetric perspiration rate.

The device comprises a zone for collecting sweat that is concave and that leads the sweat of the skin towards an inlet. The inlet is in fluid communication with a microfluidic channel that ends in an outlet or a tank for collecting sweat.

The sensor of conductivity of the sweat comprises a plurality of conductivity electrodes that are placed on a substrate, such as a printed circuit board, and that are disposed in the microfluidic channel, preferably near the inlet.

The cutaneous conductance comprises a plurality of conductance electrodes that are arranged on the device so that the conductance electrodes come in contact with the skin of the user outside of the collection zone.

The volumetric perspiration rate sensor comprises a plurality of volumetric sweating rate electrodes that are also carried on the substrate and disposed in the microfluidic channel.

During the operation of the device, when the user starts to sweat, a sweat sample penetrates into the device at the inlet and moves inside the microfluidic channel. When the sweat flows and comes in contact with the conductivity electrode, the device measures the conductivity of the sample of sweat. Likewise, as the sweat moves in the microfluidic channel, the sweat comes in contact with the successive volumetric sweating rate electrodes, which the device interprets as sweat present at the electrode contacted. The device uses the presence of sweat at each electrode, as well as a filled volume of the microfluidic channel and a time of contact, to determine the volumetric perspiration rate. As a result, the microfluidic channel is sufficiently long to successively house the electrodes between the inlet and the outlet.

In certain embodiments of the device, each volumetric sweating rate electrode is also configured to measure the conductivity of the sweat when the sweat comes in contact with it.

Certain embodiments of the device can comprise a microthermal mass flow sensor, a pressure sensor or other suitable means for independently determining the sweating rate.

Other embodiments of the device can comprise a temperature sensor, or a temperature sensor can be incorporated into one of the conductance electrodes.

Certain embodiments are configured with a disposable microfluidic channel, while other embodiments include a reusable microfluidic channel that is cleaned between the uses, for example by removing the microfluidic channel from the device and by rinsing it with air, demineralized water or a cleaning solution.

It is understood that such a device carries out a volumetric measurement that involves a storage of the sweat in the microfluidic channel. Thus, the measurement of flow rate is carried out continuously, as the microfluidic channel is filled. However, once the latter is full, it is no longer possible to continue the measurements without emptying the microfluidic channel, which has several disadvantages: it is necessary for the collection zone to have small dimensions in order to reduce a filling speed, or it is necessary for the microfluidic channel to be long to extend the measurement, or it is necessary to have a combination of the two.

A first problem is that the use of a small collection zone combined with a long measurement microfluidic channel can lead to problems resulting from the friction exerted by walls defining the microfluidic channel on the sweat collected requiring the eccrine glands to strain more and more to emit the sweat. Moreover, the reduction of the collection zone, to several millimeters in diameter in general, leads to a very significant loss of accuracy. Furthermore, by reducing the collection zone, an inference error is increased exponentially. Then, inevitable infiltrations of sweat coming from zones other than the collection zone are the cause of errors that greatly influence the final measurement. This problem of infiltration appears as soon as the user carries out an intense effort. If, moreover, the device is fastened with adhesives, problems of ungluing, allergy problems, problems of pockets of sweat moving under the adhesive, potentially all the way to the collection zone, are observed.

Another problem related to the devices using an inlet placed on the skin and measuring the flow rate of sweat volumetrically, by storing the sweat in a microfluidic channel that is as long as possible, lies in losses of sweat that escape from the collection zone. Indeed, the sweat filling the long microfluidic channel for measuring the flow rate is subjected to an increasing pressure. This pressure is directly supported by the eccrine glands that supply the microfluidic channel, that is to say the eccrine glands located in a particular zone close to the inlet. This particular zone is generally small, several square millimeters, and thus comprises few eccrine glands. Indeed, an inlet that is too large is incompatible with this type of measurement by storage of the sweat, since there would be too many eccrine glands and the microfluidic channel could fill too fast according to the people and their respective sweating rate. Thus, when the pressure increases during the filling of the microfluidic channel, problems of leakage at the inlet result. It is observed that the use of a volumetric measurement with storage of the sweat presupposes the use of small inlets and requires dimensionings of the microfluidic channel dependent on the sweat rate of the user, since the same channel can fill up slowly for one person and very quickly for another. At a given dimension, it is thus impossible to provide a fixed duration of use, since the latter varies in a very noticeable manner from one user to another. The only solution is to provide different dimensionings and to propose a range of devices covering the low, average and high flow rates, which greatly complexifies an industrial use of such a device.

Another problem lies in the fact that such an arrangement of the device does not allow an optimized circulation of the sweat inside the microfluidic channel.

Another problem lies in the fact that such a device is not adapted to carry out at least one measurement per minute in the microfluidic channel.

The document WO 2021/099610 describes devices and methods for measuring the sweating rate of a subject using a portable system. A perspiration rate can be automatically determined on the basis of one or more signals produced by a wetting sensor module in response to a presence of sweat in the portable system. The signal(s) can be produced using a device for monitoring the presence of sweat, comprising for example two electrodes or more that are usable to carry out measurements of conductance. In certain embodiments, the drops of sweat are collected periodically by the portable system and detected individually by the wetting sensor module so that the perspiration rate is determined on the basis of the periodic detection of the drops.

The wetting sensor module can indicate whether certain modules are wet and provide an estimation of the flow rate during the filling of the system. It is possible, for example, for the device to comprise a series of electrodes installed in some or all of the modules.

By carrying out measurements of conductance between pairs of electrodes, it is possible to measure whether there is an ionic contact between them and thus whether the path between them is humid. By using various combinations of electrodes, it is thus possible to follow the progression of the fluid along the way, and thus to calculate an estimation of the flow rate by using the known geometry and the fluidic capacity of the system.

A first problem lies in the fact that these measurements are random and imprecise by being based on the presence or the absence of drops, which is rather revealing of a sweating or of an absence of sweating but which does not allow to precisely measure a sweat flow rate.

A second problem lies in the fact that the device endeavors to identify a sweating per pore which does not provide an overall vision of the sweating of the user.

The known devices of the prior art result in several problems that should be solved.

The present invention is situated in this context and proposes a device for analyzing sweat capable of solving the aforementioned problems. The analysis device is capable of analyzing the sweat secreted by the skin of a user against which the device is placed.

Overall, the device comprises a housing comprising a first face provided with a collection means arranged into a sweat collector of a large size that is capable of recovering a sweating emanating from several hundred pores to provide an overall vision of sweating of a user. The collection means is calibrated, shaped and dimensioned to generate a continuous flow of sweat from the sweat collected and is optimized to maintain such a continuity of the flow of sweat.

The housing also comprises a second face equipped with a microelectronic chip that is capable of measuring, in real time a inside microfluidic channel, a concentration of NaCl, then deducing therefrom a flow rate of sweat by analysis of a variation of conductance of the sweat before the latter is evacuated. This device is disposable and is clipped onto an armband allowing the reading and the sending of data via remote communication means, of the Bluetooth or similar type, linked to the microelectronic chip to a reception device, such as a mobile telephone or similar. The data transmitted is then stored on a storage means to be analyzed and to provide performance statistics.

The sweat collector includes a collection surface of between 5 cm² and 8.5 cm², preferably between 6 cm² and 7 cm², more preferably approximately 6.25 cm², plus or minus 10%, which allows to sample at least 600 eccrine glands, in particular on the ventral side of the forearm for example. The sweat collector includes a concave face bordered by a rim that surrounds a collection zone allowing a rapid and precise flow of the sweat. The rim avoids an escape of sweat out of the sweat collector which would introduce imprecision into the measurement. It is understood that the rim surrounds the collection surface. The concave face allows a fast collection of the sweat and the formation of a regular flow of sweat. The rim and the collection surface define a determined collection volume, which is approximately 500 mm³, +/−10%.

The armband exerts a suitable bearing force on the sweat collector so that, via the rim several hundred microns high, the collection zone is perfectly isolated from the rest of the body. As a result, the sweat cannot cross the rim either to enter the collection zone or to exit therefrom. This results in a fitting pressure of the collection of sweat with respect to the collection surface. In other words, the sweat collected by the device comes only and in totality from the collection surface of the device. Indeed, the slightest contamination of the collection zone can have very significant repercussions in terms of an inference of the collection carried out. Thus, the sweat collector has a concavity and reliefs allowing the fast flow of the sweat. The concavity is developed in such a way that a force of closing of the armband is transmitted to the rim of the sweat collector which exerts a pressure on the skin of the user. This pressure results in a bead of skin to which the sweat collector conforms, without crushing the bead of skin in order to allow a phenomenon of capillarity. The sweat collector comprises hydrophobic zones that allow to channel the sweat that appears under the sweat collector and improve and channel a capillary movement of sweat towards a central orifice provided in the collection zone.

These arrangements allow to not crush the bead of skin surrounded by the rim forming because of the closing of the armband to not smother the eccrine glands by a mechanical pressure that is too great.

These arrangements allow nevertheless to also impose an additional hydraulic pressure to generate the continuous flow of sweat in order to measure a flow rate of sweat by variation in conductance, while avoiding storing sweat.

For this purpose, a ratio between a sampled collection volume, a number of eccrine glands present in the collection zone, a minimum flow rate of sweat and a measurement frequency is evaluated, given the dimensions of the microfluidic channel and the dimensions of measurement electrodes used.

The use of the sweat collector with a rim advantageously allows to sample a large surface area of skin and obtain the sweat coming from more than 600 eccrine glands. First of all, the volume of sweat collected is considerably increased because of this even during a small effort involving little sweating, approximately 25 cl/hour, an average minimal flow rate of approximately 0.04 ml/hour (or 40 mm³/hour). Such a flow rate is sufficient to fill the microfluidic channel with a volume of 7.4 mm³ of sweat in approximately 10 minutes or for a microfluidic channel having a cross-section of 0.1 mm×0.9 mm, the whole channel, which thus allows to have a first measurement of concentration and of flow rate in approximately 10 minutes after the beginning of the sweating, then to have a new series of measurements approximately every minute (new measurements independent of the preceding ones since a new layer of sweat covers each of the two pairs of electrodes), even for slight sweating. It should also be noted that in the case of a sweating in vapor phase, corresponding to a physical activity at rest, this device (in particular via the dimensional ratios between the collection zone and the microfluidic channel), allows the condensation of the sweat in the collection zone which thus flows in liquid phase in the microfluidic channel allowing to obtain measurements over time scales of approximately 30 minutes to one hour (for sweat flow rates at rest between 10 cl/hour and 25 cl/hour).

In order to have a good measurement at an affordable cost, the collection of a surface area covering at least 600 eccrine glands is essential, this allows to use a microfluidic channel having a passage cross-section of 0.1 mm×0.9 mm and a channel length of approximately 60 mm+/−10%, and to insert therein two pairs of curved electrodes 10 to 20 mm long for a height going from 30 to 40 microns and a width going from 200 to 300 microns. This results in an optimization of the sweat collector, from the size of the microfluidic channel to the dimensioning of the measurement electrodes and to the flow rate of sweat.

It is understood that without a measurement of the flow rate, the armband could not give overall information regarding the body of the user. Indeed, it is the statistical inference of the results of the sampled zone of the body that is relevant. This inference owes its accuracy to the measurement carried out on the sample and the flow rate is the most complicated measurement to carry out since the sweat is not stored.

For these reasons, it is advantageously proposed by the present invention to carry out a measurement based on a variation in conductance in the microfluidic channel. Thus, the measurement is carried out during the moment at which the sweat flows continuously in the microfluidic channel, before its ejection. It is noted that no storage of the sweat is carried out, the sweat flowing from an inlet orifice towards an outlet orifice, thus solving the problems of dimensioning of the microfluidic channel according to the flow rate of sweat.

Such a type of measurement can be carried out by the device of the present invention for several reasons taken alone or in combination. The device has a large collection zone dimensioned to generate a continuous flow of sweat, the device has a rim that surrounds the collection zone ensuring a sealing between the collection zone and the outside of the collection zone, which avoids losses of sweat. The collection zone forms a pump capable of generating the continuous flow of sweat. Thus, it was possible to observe on a user at rest having little sweating the formation of a continuous flow of sweat, in particular by condensation inside the microfluidic channel, because of its dimensions and its arrangement. The device has at least one microfluidic channel having a size and a shape suitable for facilitating a laminar flow of the sweat minimizing possible disturbances, such as a phenomenon of diffusion, inside the microfluidic channel and the device includes at least two pairs of electrodes having suitable dimensions. These features reinforce a reliability of the measurements. Thus, the collection volume and the volume of the microfluidic channel have been determined precisely ensure a continuous flow of sweat successively immersing the two pairs of electrodes. It is all of these points that allow to carry out the measurement that will be described below.

It is understood that on the basis of a coherent choice of the features of the device comprising the volume of the collection zone, the geometry shaping of the microfluidic channel, the dimensions of the microfluidic channel and the dimensions of the pairs of electrodes, such as their respective length in particular, it is possible to measure with accuracy the flow rate of sweat.

First of all, the large collection zone, containing at least 600 eccrine glands, allows to collect a significant flow rate of approximately 0.1 ml/hour at minimum. The choice was made to dimension the collection zone so that the collection zone has a collection volume of 500 mm³, +/−10%.

Secondly, the microfluidic channel houses at least two pairs of electrodes capable of measuring a conductance of the sweat, including a first pair of curved electrodes disposed upstream of a second pair of curved electrodes inside the microfluidic channel. The two electrodes are separated by an electrode distance, and each measure the conductance of the sweat at a period of time Δt according to a flow speed. Each time that the conductance of the liquid changes, a new measurement of the flow rate is carried out. The choice was made to dimension the volume of the microfluidic channel so that the microfluidic channel has a channel volume of 5.4 mm, +/−10%.

During the flow of the sweat, the first pair of electrodes measures a first conductance C₁, C₂, . . . Cₙ, the Cᵢ being spaced apart by a measurement frequency Emes, for example 1 min (C₂ is thus measured with a minute interval with respect to Cᵢ and it should be noted that possibly C₁=C₂, etc.).

The second pair of electrodes is spaced apart by the non-zero electrode distance from the first pair of electrodes, and the second pair of electrodes is strictly identical to the first pair of electrodes. The second pair of electrodes measures a second conductance C′₁, C′₂, . . . . C′ₙ, the C′ᵢ being spaced apart by the same measurement frequency Fₘₑₛ, for example 1 min (C′₂ is thus measured with a minute interval with respect to C′₁ and it should be noted that possibly C′₁=C′₂, etc.).

The second pair of electrodes measures the conductance of the continuous flow of sweat and the time at which the second conductance is equal to the first conductance is noted. The choice was made to observe this matching in measurement for the first pair of electrodes and for the second pair of electrodes to deduce therefrom the sweat flow rate in a precise manner, the flow being laminar and continuous inside the microfluidic channel.

Thus, the second pair of electrodes operates continuously, to determine the period of time Δt₁, Δt₂, . . . . Δtₙ that separates the measurements of conductance C₁, C₂, . . . . Cₙ of the first pair of electrodes and the measurements of conductance C₁, C₂′, . . . . Cₙ′ of the second pair of electrodes, which are such that C₁′=C₁, C₂′=C₂ . . . . Cₙ′=Cₙ given that the measurement of conductance Cᵢ′ was carried out with a time interval Δt₁ with respect to the measurement of conductance Cᵢ, i=1 . . . n. As a result, Δt₁ is the period of time that the continuous flow of sweat having a conductance Cᵢ takes to cover the electrode distance D that separates the two electrodes.

In other words, it is on the basis of an equivalence in conductance measured by the second pair of electrodes with respect to the first pair of electrodes, that is to say when the continuous flow of sweat having a given concentration of NaCl seen by the second pair of electrodes corresponds to that previously measured by the first pair of electrodes, that it is possible to deduce that it is the same portion of the continuous flow of sweat that has successively crossed the first pair of electrodes, then the second pair of electrodes, and thus to precisely determine a flow rate of sweat, this precision being obtained from the chosen dimensions of the microfluidic channel and of the collection zone as well as the chosen shaping of the microfluidic channel, on the basis of a continuous laminar flow with minimized disturbances because of these dimensions and shapings.

These arrangements are such that this flowmeter is purely based on the quality of the electrodes and the fact that in a microfluidic situation the phenomenon of diffusion is reduced. In other words, if the flow of sweat increases in concentration from 30 to 32 mmol/liter, in the microfluidic channel there will be a section of sweat at 30 mmol/liter and another section of sweat having a concentration of 32 mmol/liter, the two sections being separated, in the microfluidic channel, by a thin front. Because of the phenomenon of diffusion existing in a microfluidic channel which is reduced via the spiral shape of the channel, it is understood that according to the proximity of the second pair of electrodes the measurement of 32 mmol/liter recorded by the first pair of electrodes is encountered again.

For this purpose, the method of the present invention is a method for analyzing a sweat emitted by a skin of a user, the method comprising a first step of collecting the sweat emitted by the skin of the user via a collection means that a first face of a housing comprises to form a continuous flow of sweat.

The method comprises a second step of bringing the continuous flow of sweat collected towards a means for analyzing the continuous flow of sweat that a second face of the housing provided with a microfluidic channel comprises.

The method comprises a third step of measurement of a first conductance of the continuous flow of sweat by a first pair of electrodes housed inside the microfluidic channel.

The method comprises a fourth step of measurement of a second conductance of the continuous flow of sweat by a second pair of electrodes housed inside the microfluidic channel and placed at an electrode distance from the first pair of electrodes.

The method comprises a fifth step of determining a period of time passed for the second conductance to be equal to the first conductance.

The method comprises a sixth step of calculating a flow rate of sweat produced by the skin of the user.

The device for implementing such a method comprises a housing that includes a first face provided to be in contact with the skin of the user. The first face is equipped with a means for collecting the sweat comprising a collection zone and a collection volume. The housing includes a second face that is equipped with a means for analyzing the sweat that comprises at least one microfluidic channel having a channel volume and housing a first pair of electrodes capable of measuring a first conductance of the sweat, and a second pair of electrodes capable of measuring a second conductance.

The device advantageously comprises at least any one of the following technical features, taken alone or in combination:
a ratio between a collection volume and the volume of the channel is between 80 and 110, preferably approximately 90+/−10%,
the microfluidic channel successively comprises a first linear portion, then a first semi-circular portion, then a second linear portion, then a second semi-circular portion, then a third linear portion,
the second semi-circular portion houses the pairs of electrodes that are curved,
the second semi-circular portion comprises a first half of second semi-circular portion that houses the first pair of electrodes and a second half of second semi-circular portion that houses the second pair of electrodes,
the microfluidic channel has a rectangular cross-section,
the rectangular cross-section of the microfluidic channel is approximately 0.09 mm², +/−10%,
the collection zone is surrounded by a rim,
the collection means includes at least one hydrophobic zone,
the housing consists of a one-piece assembly,
the housing comes from molding of a plastic material,
the housing is equipped with a microelectronic chip that comprises calculation means and that is associated with communication means,
the device comprises at least one armband that is attached onto a support housing the housing to maintain the first face against the skin of the user and to ensure a contact on the skin of the user of the collection means with a pressure greater than 15 g per cm².

Other features and advantages of the invention will also appear through the following description on the one hand and through several exemplary embodiments given for illustrative and non-limiting purposes in reference to the appended drawings on the other hand, which are briefly described below:

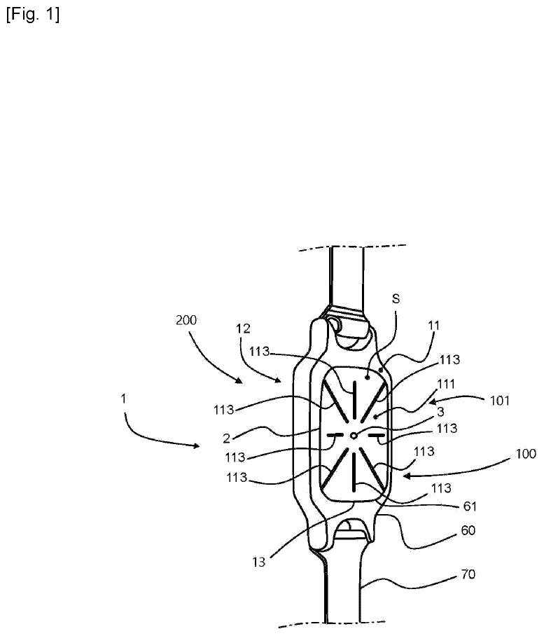

FIG. 1 is a profile diagram of an analysis device according to the present invention,

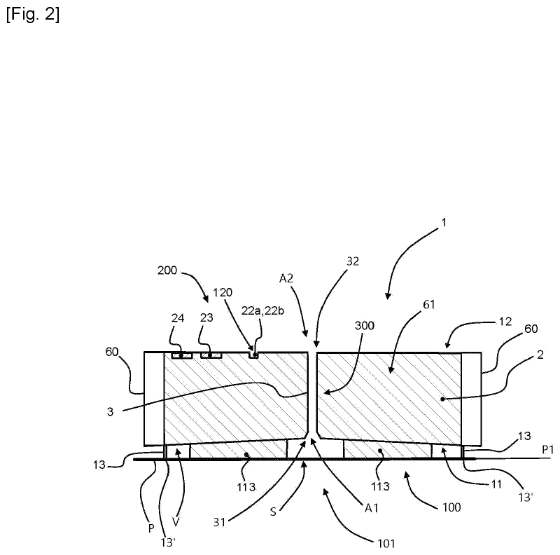

FIG. 2 is a diagram of a median cross-sectional view of a housing that is part of the analysis device illustrated in FIG. 1,

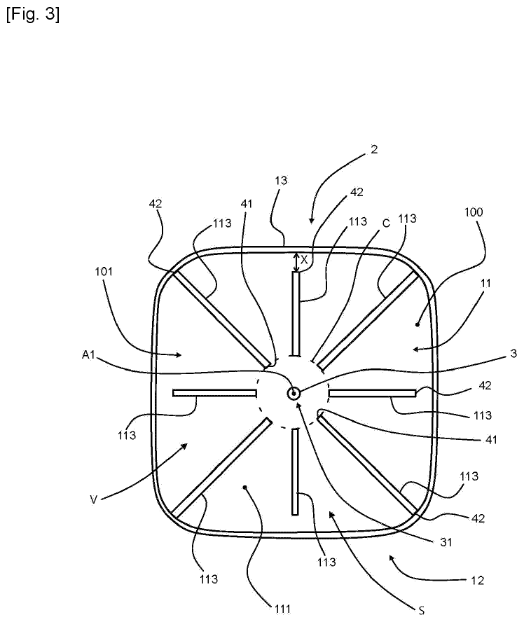

FIG. 3 is a diagram of a bottom view of the housing shown in FIG. 2 illustrating a collection means of said device,

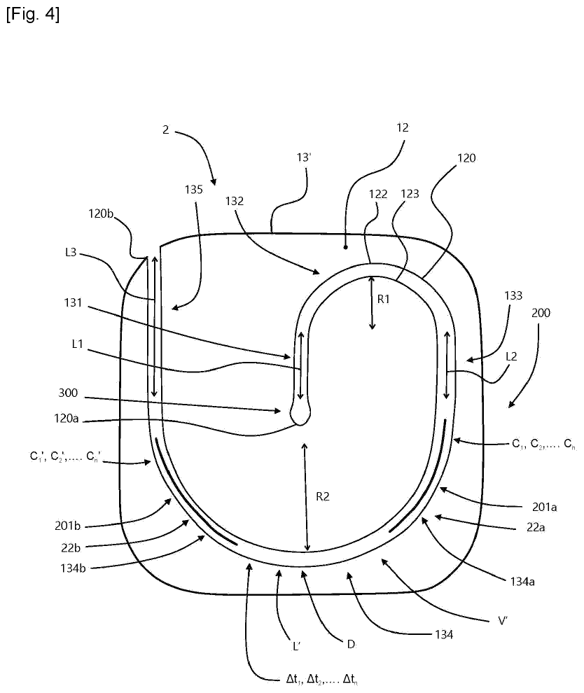

FIG. 4 is a diagram of a top view of a preferred alternative embodiment of the housing shown in FIGS. 2 and 3.

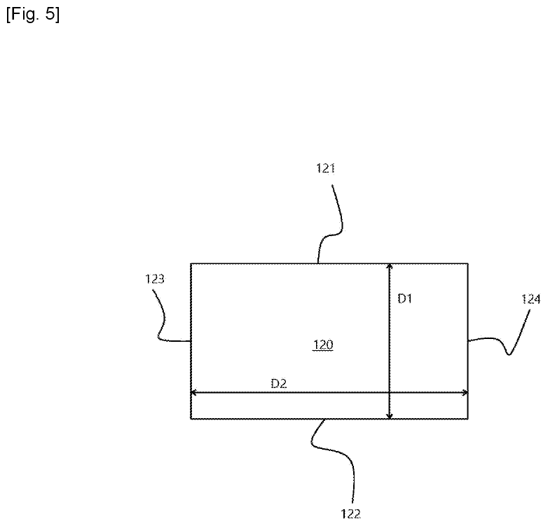

FIG. 5 is a diagram of a cross-sectional view of the microfluidic channel.

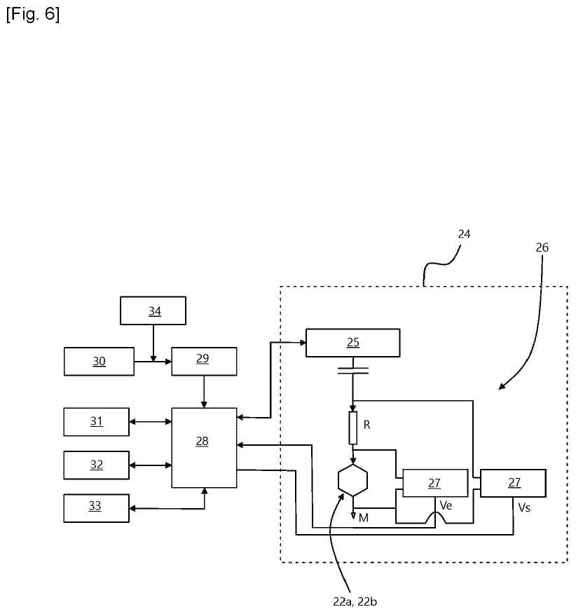

FIG. 6 is a diagram of a measuring block that is part of the device illustrated in FIG. 1.

FIG. 1 and FIG. 2 show a device 1 intended to analyze a sweat present on a skin P of a user. This is understood to mean that the device 1 is portable and is adapted to analyze the sweat generated by eccrine glands that the skin P of the user comprises during a physical effort, the user being for example an athlete participating in a sporting event, or a patient, the analysis of the sweat of which can be medically relevant. For this purpose, the device 1 comprises an armband 70, or a bracelet for maintaining the device 1 on the user, for example on an arm, a leg or the torso of the latter. It is noted that the armband 70 is configured so that the skin P of the user forms a bead under the effect of the pressure exerted by the device 1 on the skin P of the user. In particular the armband is capable of ensuring a contact on the skin of the user of the collection means with a pressure greater than 15 g per cm². More particularly, the device 1 is arranged to be clipped, fitted, or fastened by any other maintaining means on a support 60 that is provided with the armband 70.

The support 60 includes an opening 61 that is capable of housing a housing 2 that is part of the device 1. The housing 2 includes a first face 11 provided to be in contact with the skin P of the user and a second face 12, preferably opposite to the first face 11 and more preferably parallel to the first face 11. It is understood that the opening 61 opens through the housing 2 so that the first face 11 is in contact with the skin P of the user.

The housing 2 consists of a one-piece assembly in the sense that the housing 2 is formed by an assembly that cannot be taken apart, unless by an alteration or even a destruction of the housing 2. For this purpose, the housing 2 for example comes from molding of a plastic material in particular.

In FIG. 3, the first face 11 is equipped with a means 100 for collecting the sweat that comprises a collection zone 101 having a collection surface S. The collection zone 101 is part of the first face 11 and is comprises inside a rim 13 that the first face 11 comprises. The rim 13 is arranged so that, during a bearing exerted by the armband 70 on the device 1, the skin P of the user forms a bead facing the collection zone 101. Moreover, the rim 13 is arranged to prevent an evacuation of the sweat produced by the bead out of the collection zone 101 and to prevent an intake of sweat produced outside of the collection zone 101 into the latter. In other words, the rim 13 is shaped into a barrier of the collection zone 101 through which the sweat cannot pass. It is understood that the collection zone 101 is arranged to recover the sweat produced by the eccrine glands of the bead of the skin P and that the collection zone 101 has the collection surface S expressed in mm². For example, the collection surface S is for example approximately 6.25 cm², +/−10%, if the collection zone 101 is substantially shaped into a square with sides of 2.5 millimeters. Moreover, the collection zone 101 has a collection volume V that is approximately 500 mm³, +/−10%. It is understood that the collection volume V is defined by the first face 11, the rim 13 and a plane P1 in which a free border 13′ of the rim 13 is inscribed, the free border 13′ of the rim 13 being provided to be in contact with the skin P of the user.

The second face 12 is equipped with a means 200 for analyzing the sweat that comprises at least one microfluidic channel 120, as illustrated in FIGS. 4 and 5.

The collection means 100 and the analysis means 200 are connected via a supply means 300, more particularly visible in [FIG. 2], which is arranged into a linking means between the collection means 100 and the analysis means 200, by connecting the first face 11 and the second face 12. According to a preferred embodiment, the supply means 300 comprises at least one duct 3 that extends for example orthogonally to the first face 11 and to the second face 12. The duct 3 extends between an inlet orifice 31 provided in a first center A1 of the first face 11 and an outlet orifice 32 provided in a second center A2 of the second face 12.

The collection means 100 includes at least one relief 113 that forms a ramp for guiding the sweat, the sweat tending to flow along the relief 113 towards the first center A1.

Preferably, there is a plurality of the reliefs 113 and they extend radially from the inlet orifice 31 towards the peripheral rim 13. More particularly, each relief 113 extends between a first end 41 placed on a circle C arranged around the first center A1 and a second end 42. Certain reliefs 113 include a second end 42 that can be part of the peripheral rim 13, while other reliefs 113 include a second end 42 that is arranged at a non-zero end distance X from the rim 13. It is noted that the reliefs 113 indifferently have a parallelepipedic shaping, sinusoidal shaping or similar.

The collection means 100 preferably includes at least one hydrophobic zone 111. The hydrophobic zone 111 is for example obtained by laser nanostructuring of the first surface 11 of the housing 2.

The collection means 100 is preferably concave, so as to facilitate a flow of the sweat from the hydrophobic zone 111 towards the first center A1 which forms the low point of the concavity of the collection zone 101. To further facilitate such a flow, the inlet orifice 31 is arranged in the shape of a funnel.

In FIG. 4, the analysis means 200 comprises the microfluidic channel 120 fluidly connected to the hydrophobic zone 111 via the supply means 300. The microfluidic channel 120 extends between an intake 120a provided in the duct 3 and an evacuation 120b provided in a peripheral border 13′ of the second face 12. Between the intake 120a and the evacuation 120b, the microfluidic channel 120 has a channel length L′ that is approximately 60 mm, +/−10%. The microfluidic channel 120 includes at least two measurement zones 201a, 201b capable of each housing at least one pair of electrodes 22a, 22b.

In reference also to [FIG. 5], the microfluidic channel 120 has a rectangular cross-section. The microfluidic channel 120 is defined by an upper wall 121 and a lower wall 122 spaced apart from one another by a first distance D1 that is approximately 100 μm+/−10%. The microfluidic channel 120 is also defined by a first lateral wall 123 and a second lateral wall 124 spaced apart from one another by a second distance D2 that is approximately 900 μm+/−10%. Such a shaping of the microfluidic channel 120 is a deliberate choice that allows in particular a laminar flow, free from turbulence and disturbance, to the sweat that circulates inside the microfluidic channel. The microfluidic channel 120 has a volume of the channel V′ which extends between the intake 120a and the evacuation 120b and which is located at the end of the microfluidic channel 120 so that the volume of the channel V′ from the inlet point to the outlet point is between 5 and 6 mm³, preferably equal to 5.4 mm³.

In [FIG. 4], the microfluidic channel 120 successively comprises a first linear portion 131 having a first length L1 that is approximately 4.4 mm+/−10%, then a first semi-circular portion 132, the first lateral wall 123 of which is inscribed in a first arc of a circle having a first radius of curvature R1 that is approximately 4.6 mm+/−10%, then a second linear portion 133 having a second length L2 that is approximately 5 mm+/−10%, then a second semi-circular portion 134, the first lateral wall 123 of which is inscribed in a second arc of a circle having a second radius of curvature R2 that is approximately 4.6 mm+/−10%, then a third linear portion 135 having a third length L3 that is approximately 11.6 mm+/−10%. Such a spiral shaping of the microfluidic channel 120 is also a deliberate choice that provides a guarantee of laminar flow, free from turbulence and disturbance, to the sweat that circulates inside the microfluidic channel. As a result, the microfluidic channel has a channel length L′ that is approximately 60 mm, +/−10%.

The two pairs of electrodes 22a, 22b are curved and are distributed the inside second semi-circular portion 134. More particularly, the second semi-circular portion 134 comprises a f first half of second semi-circular portion 134a that houses the first pair of electrodes 22a and a second half of second semi-circular portion 134b that houses the second pair of electrodes 22b.

Thus, and advantageously, a ratio between the collection volume V and the volume of the channel V′ is between 80 and 110, preferably approximately 92.5+/−10%.

Such a ratio provides an optimized circulation of the sweat inside the microfluidic channel 120. Thus, such a device 1 is adapted to carry out at least one measurement per minute in a microfluidic channel 120 including a passage cross-section smaller than 0.1 mm², and in particular a passage cross-section of approximately 0.09 mm²+/−10%, the microfluidic channel 120 having a length of approximately 6 cm+/−10%.

Preferably, there is a plurality of pairs of electrodes 22a, 22b and they are disposed in distinct measurement zones 201a, 201b, the pairs of electrodes 22a, 22b are indifferently selective or non-selective to obtain data relative to a hydric loss; a concentration of ions; a concentration of Na⁺ ions; a concentration of Cl— ions; a concentration of lactate; a temperature and/or a loss of calories.

The microfluidic channel 120 comprises a first measurement zone 201a that houses a first pair of electrodes 22a such as a pair of electrodes intended to measure a conductance of the sweat.

The microfluidic channel 120 comprises a second measurement zone 201b that houses a second pair of electrodes 22b identical to the first pair of electrodes 22a.

The first pair of electrodes 22a and the second pair of electrodes 22b are spaced apart from one another by a non-zero electrode distance D taken inside the microfluidic channel 120.

In FIG. 6, to measure the concentration of NaCl present in the continuous flow of sweat and determine the flow rate of sweat, two measurement blocks 24 were carried out with a voltage generator 25, a divider bridge 26 and two converters 27 of the Vrms/DC type. The output voltages Ve of the converters 27 are then sent to a single microcontroller 28 that is part of an electronic chip 23, visible in FIG. 2.

The voltage generator 25, equipped with a condenser, is set to 200 mV and to 200 KHz to power the divider bridge 26.

The input of the divider bridge starts with a resistance R of 400Ω and ends with a screen-printed electrode, having a validatable and NaCl-dependent conductivity, before reaching the ground M.

A first Vrms to DC converter is used to read the voltage at the terminals of the voltage generator Ve and a second Vrms to DC converter is used to read the voltage at the terminals of the variable electrode Vs. The voltages at the output of the converters 27 are sent back towards the microcontroller 28.

Preferably, the second face 12 of the housing 2 houses the microelectronic chip 23 as well as the measurement blocks 24 linked to the electrodes 22a, 22b housed inside the measurement zones 201a, 201b. The microelectronic chip 23 comprises calculation means capable of carrying out the determination and calculation steps of the method described below.

These calculation means are in particular capable of deducing the conductance Ci of the continuous flow of sweat from the following relationship:

$$Ci = \frac{\frac{{V}_{e}}{{V}_{s}} - 1}{R}$$

Then, a concentration X of NaCl is deduced from the following formula:

$$X = 0.0868 {Ci}^{2} + 5.4606 Ci - 0.097$$

It is noted that the microcontroller 28 is linked to a voltage regulator 29, a battery charger 30, an accelerometer 31, a temperature sensor 32 and a humidity sensor 33, the voltage regulator 29 and the battery charger 30 being linked to an accumulator 34.

The microelectronic chip 23 is for example linked to at least one NFC/RFID chip or physical connectors (studs or connection socket), a rechargeable microbattery and a Bluetooth module that are provided in the support 60.

According to one embodiment, the device 1 comprises means for memory of said data.

These arrangements are such that the device 1 is capable of implementing an analysis method of the present invention.

More particularly, the analysis method of the present invention comprises:
a first step of collecting the sweat emitted by the skin P of the user via a collection means 100 that the first face 11 of the housing 2 comprises to form a continuous flow of sweat,
a second step of bringing the continuous flow of sweat collected towards a means 200 for analyzing the continuous flow of sweat that the second face 12 of the housing 2 provided with the microfluidic channel 120 comprises,
a third step of measurement of the first conductance C₁, C₂, . . . . Cₙ of the continuous flow of sweat by the first pair of electrodes 22a housed inside the microfluidic channel 120,
a fourth step of measurement of the second conductance C₁′, C₂′, . . . . Cₙ′ of the continuous flow of sweat by a second pair of electrodes 22b housed inside the microfluidic channel 120 and placed at an electrode distance D from the first pair of electrodes 22a,
a fifth step of determining a period of time Δt₁, Δt₂, . . . Δtₙ passed for the second conductance C₁′, C₂′, . . . Cₙ′ to be equal to the first conductance C₁, C₂, . . . Cₙ, and
a sixth step of calculating a flow rate of sweat produced by the skin P of the user.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. Device for analyzing a sweat emitted by a skin of a user, the device comprising a housing that includes a first face provided to be in contact with the skin of the user, the first face being equipped with a means for collecting the sweat comprising a collection zone and a collection volume, the housing including a second face that is equipped with a means for analyzing the sweat that comprises at least one microfluidic channel having a channel volume housing a first pair of electrodes capable of measuring a first conductance of the sweat, and a second pair of electrodes capable of measuring a second conductance, wherein the microfluidic channel is shaped into a spiral and successively comprises a first linear portion, then a first semi-circular portion, then a second linear portion, then a second semi-circular portion, then a third linear portion, the second semi-circular portion housing the pairs of electrodes that are curved, the second semi-circular portion comprising a first half of second semi-circular portion that houses the first pair of electrodes and a second half of second semi-circular portion that houses the second pair of electrodes.

16. Device according to claim 15, wherein a ratio between the collection volume and the volume of the channel is between 80 and 110.

17. Device according to claim 15, wherein the microfluidic channel has a rectangular cross-section.

18. Device according to claim 17, wherein the rectangular cross-section of the microfluidic channel is approximately 0.09 mm², +/−10%.

19. Device according to claim 15, wherein the collection zone is surrounded by a rim.

20. Device according to claim 15, wherein the collection means includes at least one hydrophobic zone.

21. Device according to claim 15, wherein the housing comprises a one-piece assembly.

22. Device according to claim 15, wherein the housing comes from molding of a plastic material.

23. Device according to claim 15, wherein the housing is equipped with a microelectronic chip that comprises calculation means and that is associated with communication means.

24. Device according to claim 15, wherein the device comprises at least one armband that is attached onto a support housing the housing to maintain the first face against the skin of the user and to ensure a contact on the skin of the user of the collection means with a pressure greater than 15 g per cm².

25. Method for analyzing a sweat emitted by a skin of a user using an implementation of a device according to claim 15, the method comprising a first step of collecting the sweat emitted by the skin of the user via a collection means that a first face of the housing comprises to form a continuous flow of sweat, a second step of bringing the continuous flow of sweat collected towards a means for analyzing the continuous flow of sweat that a second face of the housing provided with a microfluidic channel comprises, the method comprising a third step of measurement of a first conductance of the continuous flow of sweat by a first pair of electrodes housed inside the microfluidic channel and a fourth step of measurement of a second conductance of the continuous flow of sweat by a second pair of electrodes housed inside the microfluidic channel and placed at an electrode distance from the first pair of electrodes, the method comprising a fifth step of determining a period of time passed for the second conductance to be equal to the first conductance and a sixth step of calculating a flow rate of sweat produced by the skin of the user.
